(19) United States
(12) Patent Application Publication
(10) Pub. No.: US 2026/0102428 A1
(43) Pub. Date: Apr. 16, 2026

(54) COMPOSITIONS AND METHODS FOR THE INHIBITION OF METHICILLIN RESISTANT STAPHYLOCOCCUS AUREUS

(71) Applicants: THE UAB RESEARCH FOUNDATION, BIRMINGHAM, AL (US); UNIVERSITY OF KANSAS, Lawrence, KS (US)

(72) Inventors: Olaf KUTSCH, Birmingham, AL (US); Rachel ANDREWS, Birmingham, AL (US); Susan BIRKET, Pelham, AL (US); Stefan H. BOSSMANN, Fairway, KS (US)

(21) Appl. No.: 19/134,788

(22) PCT Filed: Dec. 4, 2023
(86) PCT No.: PCT/US2023/082225

§ 371 Date: Jun. 2, 2025

Related U.S. Application Data
(60) Provisional application No. 63/386,686, filed on Dec. 9, 2022.

(51) Int. Cl. A61K 33/30; A61K 9/06; A61K 31/12; A61K 31/7048; A61K 33/34; A61K 47/10; A61K 47/26; A61P 31/04

(52) U.S. Cl. CPC A61K 33/30; A61K 9/06; A61K 31/12; A61K 31/7048; A61K 33/34; A61K 47/10; A61K 47/26; A61P 31/04

(57) ABSTRACT

In one aspect, the disclosure relates to a method for treating or preventing a bacterial infection in a subject, the methods including at least the step of administering a composition including a transition metal and avobenzone to the subject. In one aspect, the transition metal can be present as a transition metal salt such as, for example, ZnSO₄. In a further aspect, the bacterial infection can be caused by any pathogenic bacteria, including, but not limited to, Gram-positive bacteria such as, for example, methicillin-resistant Staphylococcus aureus (MRSA). In one aspect, the compositions can be applied topically in the forms of gels, creams, lotions, sprays, and the like. In any of these aspects, the compositions are non-toxic to mammalian cells. Also disclosed are compositions that can be used to perform the disclosed methods.

### CROSS REFERENCE TO RELATED APPLICATIONS

This application claims the benefit of U.S. Provisional Application No. 63/386,686 filed on Dec. 9, 2022, which is incorporated herein by reference in its entirety.

### STATEMENT REGARDING FEDERALLY SPONSORED RESEARCH OR DEVELOPMENT

This invention was made with government support under R01Al121364 awarded by the National Institutes of Health. The government has certain rights in the invention.

### BACKGROUND

As a result of the worldwide rise of antibiotic-resistant bacteria and the simultaneous decline in antibiotic drug discovery, antibiotic-resistant pathogens represent an imminent global health emergency. The increased incidence of infections with antibiotic-resistant bacteria results in longer hospital stays, increased treatment costs, and in higher numbers of patients succumbing to bacterial infections. In 2019, the six primary pathogens for which patient death was associated with antibacterial resistance were Escherichia coli, Klebsiella pneumoniae, Streptococcus pneumoniae, Acinetobacter baumannii, Pseudomonas aeruginosa, and Staphylococcus aureus. Together, drug resistant forms of these bacteria were responsible for ˜900 000 deaths worldwide with a total of ˜3.5 million deaths being attributable to antimicrobial resistance. Methicillin-resistant S. aureus (MRSA) alone caused >100,000 deaths in 2019 and is considered as especially problematic.

Despite the ongoing antimicrobial resistance crisis, antibiotic development has largely stalled. Only two new antibiotic classes have been discovered since the 1970s, and of the few antibiotics brought to market, the majority were initially identified during the Golden Age of antibiotic discovery between the 1940s and 1970s but were not approved for clinical use until the 21^{st} century. Since the fall of the Waksman platform that fueled the Golden Age, modern high throughput screening attempts have focused on identifying inhibitors of known protein targets, such as DNA gyrase, but have eliminated whole-cell screening methods that take bacterial permeability and metabolism into account. Attempts to chemically modify scaffolds of synthetic or natural antibiotics to improve potency, increase the activity spectrum, and overcome mechanisms of drug resistance have governed antibiotic drug discovery for the last few decades. These approaches helped lower the cost of drug development as the bacterial targets had already been classified and side effects were known, which accelerated the approval process. However, the repeated introduction of only slightly modified drugs (e.g. β-lactams), chemical entities for which bacteria had already developed resistance mechanisms, provided only temporary relief. Other approaches, such as the use of phages, polymers, and engineered nanoparticles have certainly shown promise, but clinical feasibility of these therapy candidates for standard clinical use remains still to be proven. Overall there has been a decades-long antibacterial innovation gap, with the exception of the recent discovery of teixobactin, an antibiotic that was identified in soil samples and established a new class of antibiotics.

Another avenue of antibiotic drug discovery is the identification and advancement of metal-dependent inhibitors, small molecule inhibitors that only exert their activity in the presence of a transition metal. These efforts have primarily focused on Cu-dependent inhibitors on the premise that the inherent antibacterial and self-potentiating redox cycling of Cu could be focused against a target organism. Indeed, this very property has been harnessed by innate immune cells where Cu is actively imported into phagosomes to damage engulfed pathogens. Unfortunately, the nonspecific cytotoxic activity of these inhibitors, which seems to be related to the redox activity of the copper component, poses significant challenges to their development as their activities also target eukaryotic cells, resulting in unacceptable therapeutic indices.

However, there is still a scarcity of antibiotic compositions that are potent and efficacious against pathogens, including methicillin resistant strains of S. aureus, while also being non-toxic to eukaryotic cells. There is further a need for new methods to treat and/or prevent skin infections as well as infections of wounds, surgical incisions, burns, and the like, using the compositions. These needs and other needs are satisfied by the present disclosure.

### SUMMARY

In accordance with the purpose(s) of the present disclosure, as embodied and broadly described herein, the disclosure, in one aspect, relates to a method for treating or preventing a bacterial infection in a subject, the methods including at least the step of administering a composition including a transition metal and avobenzone to the subject. In one aspect, the transition metal can be present as a transition metal salt such as, for example, ZnSO₄. In a further aspect, the bacterial infection can be caused by any pathogenic bacteria, including, but not limited to, Gram-positive bacteria such as, for example, methicillin-resistant Staphylococcus aureus (MRSA). In one aspect, the compositions can be applied topically in the forms of gels, creams, lotions, sprays, and the like, or can be administered orally, by injection, or intravenously. In one aspect, an oral dosage form can include a tablet, capsule, pill, powder, granule, suspension, syrup, emulsion, or any combination thereof. In any of these aspects, the compositions are non-toxic to mammalian cells. In one aspect, the pathogenic bacteria do not become resistant to the disclosed compositions and methods over time. Also disclosed are compositions that can be used to perform the disclosed methods.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1C show metal-dependent screening identifies metal-activated antibiotic against MRSA. (FIG. 1A) The bioactives library from Selleckchem was screened in 1×RPMI 1640 with and without 25 μM CuSO₄ or 25 μM ZnSO₄ against the MRSA strain USA300-LAC. Growth was measured indirectly by the conversion of the metabolic dye, reisazurin, to the fluorescent product resorufin (ex/em 530/590 nm) after overnight incubation at 37° C. with 5% CO₂. Compounds that reduced the growth of USA300-LAC by at least 50% were declared as hits. This screen outline was generated using BioRender.com. (FIG. 1B) Compounds were classified into various activity groups by graphing the percentage growth of USA300-LAC in the presence of each compound with a metal to that without the same metal. Inactive compounds are displayed in the upper right quadrant, while independent compounds plot to the lower left quadrant. The upper left quadrant represent metal-dependent hits, and inverse hits are shown in the lower right quadrant. Zn-dependent screening identified 49 Zn-dependent hits, 79 independent hits, and 3 Zn-inverse hits. (FIG. 1C) Cu-dependent screening identified 37 Cu-dependent hits, 80 independent hits, and 2 Cu-inverse hits.

FIG. 2A shows chemical structure of avobenzone. FIG. B shows chemical structure of bacitracin. FIGS. 2C-2D show metal-binding capacity of AVB and bacitracin. Job plots of AVB with ZnCl₂ (FIG. 2C) and CuBr (FIG. 2D) were made to determine the stoichiometry of the AVB-Zn and AVB-Cu complexes using the method of continuous variation. Solutions of various molar ratios (C/Cₘₐₓ) of AVB to either Zn or Cu were incubated for 30 min, and absorbance of the solutions were measured at 580 nm. The difference in absorbance (AA) was calculated for each solution relative to a standard.

FIGS. 3A-3D show metal-specific anti-staphylococcal activities of AVB and bacitracin. The minimal inhibitory concentrations (MIC) of AVB (FIG. 3A) and bacitracin (FIG. 3B) were determined against USA300-LAC under multiple metal conditions by titrating each compound in 1×RPMI 1640 with and without either 25 μM CuSO₄ or 25 μM ZnSO₄. Growth was determined after 20 h of incubation at 37° C. using A600 measurements as a readout. Percentage growth was calculated by normalizing the A600 measurements relative to that of the compound-untreated control of each metal condition. Data represent the mean±SEM (n=3 biological replicates, 3 technical replicates each). The sensitivity of the multidrug-resistant MRSA isolates, CI-1-5, to (FIG. 3C) AVB-Zn and (FIG. 3D) bacitracin-Zn were compared to USA300-LAC using dose-response curves described earlier (FIGS. 3C-3D) with 25 μM ZnSO₄. Data represent the mean±SEM (n=3 biological replicates, 3 technical replicates each).

FIGS. 4A-4B show minimal Zn requirements of AVB and bacitracin. (FIG. 4A) AVB and (FIG. 4B) bacitracin were serially titrated against serial titrations of ZnSO₄ in 1×RPMI 1640 to determine the minimum concentration of Zn required to achieve the lowest minimal inhibitory concentration (MIC) of each compound against USA300-LAC. Bacterial growth was measured using endpoint A600 measurements after 20 h of incubation at 37° C. Growth was normalized as a percentage of the growth of the untreated control. Data are representative of three separate experiments. Individual Zn concentrations that did not result in major changes in bacterial growth are not presented for clarity.

FIGS. 5A-5D show eukaryotic toxicity of AVB-Zn. Jurkat T cells (FIGS. 5A-5B), the monocytic THP-1 cells (FIGS. 5C-5D) and human primary T cells (n=3) Absolute cell viability (FIGS. 5A and 5C) was then determined by flow cytometry as the percentage of cells within the life-gate as determined by forward scatter/side-scatter analysis. As the utilized GUAVA EasyCyte is a capillary-based flow cytometer it allows for the direct determination of absolute cell numbers (within the life-gate) which can be used to determine possible effects on cell proliferation (FIGS. 5B and 5D).

FIGS. 6A-6B show AVB-Zn efficacy in a mouse wound model of MRSA infection. (FIG. 6A) To determine the antibacterial efficacy of these lotion preparations, a wound model of S. aureus infection in C57BL/6 mice that simulates a soft tissue infection using USA300-LAC was used. Lotion treatment began 24 hours post-application of the bacteria and continued through day 7. Treatments included a vehicle control (n=10), a commercially available bacitracin-Zn ointment (n=5), bacitracin-Zn lotion (n=5), avobenzone (AVB, n=5), and avobenzone-Zn (AVB-Zn, n=5). The in vivo antibacterial effect of each treatment was determined by measuring USA300-LAC colony forming units (CFUs) isolated from the wounds and mouse survival. Animals that showed signs of severe distress were euthanized. The schematic for the mouse model was created using BioRender.com. (FIG. 6B) To determine the antibacterial efficacy of these lotion preparations a wound model of S. aureus infection in C57BL/6 mice was used. The cranial thoracodorsal region of each mouse was shaved and cleaned for surgery and a silicone O-ring was secured to the shaved area. A biopsy punch created a 6 mm wound within each O-ring and infection was established by placing and securing an 8 mm dressing coated with USA300-LAC over the wound. Lotion treatment began 24 hours post application of the bacteria. Given the dimension of the punch biopsy and the 24 hours incubation time prior to treatment, the ensuing infection would be considered equivalent to a deep tissue infection. The experiment was conducted for a total of 8 days. The antibacterial effect of avobenzone was confirmed by mouse survival. Animals that showed signs of extensive sepsis were euthanized. The data show survival percentage of animals treated with vehicle control (n=10), a commercially available bacitracin lotion preparation (n=5), avobenzone lotion (n=5) and AVB-Zn lotion (n=5).

FIGS. 7A-7D show the Zn and Cu screening results for all the antibiotics and antiseptics included in the bioactives library were plotted to confirm their expected activity against MRSA. Members of the rifamycin antibiotic class (4) were independently active against USA300-LAC regardless of Zn (FIG. 7A) or Cu (FIG. 7B). Meanwhile, narrow spectrum antibiotics specific for mycobacteria (7) were classified as inactive irrespective of metal condition (FIGS. 7C-7D).

FIGS. 8A-8B show the metal-dependent screening activities of cyclic polypeptide antibiotics are highlighted among the full spectrum of antibiotics and antiseptics screened with Zn (FIG. 8A) and Cu (FIG. 8B). Metal-dependent screening correctly identified the Zn-specific cyclic polypeptide bacitracin as possessing Zn-dependent activity but not independent or Cu-dependent action against USA300-LAC.

FIG. 9 shows colony forming units (CFUs) were isolated from the wounds of surviving mice at the end of the treatment period using sterile swabs and cells were cultured on mannitol salt agar plates overnight at 37° C. Means were calculated for each treatment condition, and significance was determined using a Kruskal-Wallis test.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein is use of zinc (Zn), a d-block transition metal commonly regarded as an essential micronutrient with no redox activity, as an antimicrobial. In one aspect, neutrophils and macrophages have been shown to employ Zn similarly to Cu against phagocytized Streptococcus pyogenes and mycobacteria, respectively. In another aspect, Zn can also activate chemical compounds to become antibacterial. In a further aspect, bacitracin, a common antibiotic in first-aid ointments, is primarily active in the presence of Zn, and PBT2, a compound in clinical trials for the treatment of Alzheimer's and Huntington's disease has been shown to be an effective inhibitor of multiple bacterial species when combined with high concentrations of Zn.

In some aspects, Zn, in contrast to Cu, or transition metals in general, is the only transition metal that does not have redox activity. In one aspect, thus, Zn-activated compounds therefore are less likely to have metal-driven toxicity.

In one aspect, identification of Zn-dependent antibiotics clearly demonstrates Zn-activated antibiotics are available within the available chemical space and therefore can expand the existing chemical space, as they render otherwise inactive compounds into potent metal-antibiotics as shown herein. Despite this, no dedicated screening effort has been employed to identify specifically Zn-activated antimicrobials. Disclosed herein are drug screens designed to discover Zn-activated inhibitors efficiently identify new antibiotics. In one aspect, a Zn-dependent screen of a commercially available library of bioactive compounds against the model organism and major animal pathogen MRSA to identify Zn-activated metallo-antibiotics effective against a drug-resistant pathogen the Centers for Disease Control and Prevention (CDC) considers a serious threat to human health. In another aspect, it is shown herein that Zn is highly efficient at activating previously unknown metal-dependent inhibitors against MRSA from a library of FDA-approved drugs and bioactive molecules. In an aspect, avobenzone (AVB), an active ingredient in sunscreens, is a potent and effective Zn-activated inhibitor of MRSA. In another aspect, Zn-activated AVB (AVB-Zn) is effective at inhibiting multi-drug-resistant MRSA isolates at concentrations with no apparent eukaryotic toxicity. In still another aspect, AVB-Zn can be developed as a therapeutic lotion as shown using a murine wound infection model of MRSA.

Disclosed herein is a method for treating or preventing a bacterial infection in a subject, the method including at least the step of administering a composition containing a transition metal and avobenzone to the subject. In one aspect, the transition metal can be copper, zinc, manganese, nickel, cobalt, silver, iron, another transition metal, or any combination thereof. In another aspect, the transition metal can be in a +1 or +2 oxidation state. In some aspects, the transition metal can be Zn(II).

In an aspect, the subject can be a mammal, bird, reptile, or amphibian, such as, for example, a human, mouse, rat, guinea pig, rabbit, dog, cat, horse, cattle, swine, goat, sheep, chicken, turkey, duck, or another pet, research animal, or livestock animal.

In another aspect, the composition can be administered topically. In one aspect, the composition can be formulated as a cream, an ointment, a paste, a gel, a lotion, a milk, a suspension, an aerosol, a non-aerosol spray, a foam, a dusting powder, a pad, a patch, or any combination thereof. In another aspect, the composition can be administered orally. In a further aspect, the composition can be formulated as tablets, capsules, pills, powder, granules, a suspension, a syrup, an emulsion, or any combination thereof. In a still further aspect, the composition can be administered intravenously or by injection.

In still another aspect, the composition is administered to a site such as, for example, the skin of a subject, a wound, a burn, a surgical incision, or any combination thereof. In one aspect, the bacterial infection can be caused by a Gram-positive bacterium such as, for example, methicillin-resistant S. aureus (MRSA). In one aspect, the Gram-positive bacterium can be resistant to bacitracin. In another aspect, the pathogenic bacteria do not become resistant to the disclosed pharmaceutical compositions.

In some aspects, in performing the disclosed methods, an antibiotic can also be administered to the subject. In one aspect, the antibiotic can be selected from amoxicillin, clavulanic acid, ampicillin, sulfobactam, penicillin, vancomycin, ceftriaxone, daptomycin, ciprofloxacin, levofloxacin, ofloxacin, clindamycin, erythromycin, gentamicin, tetracycline, sulfamethoxazole, trimethoprim, another antibiotic, or any combination thereof. In some aspects, the composition and the antibiotic are administered sequentially. In other aspects, the composition and the antibiotic are administered simultaneously.

In any of these aspects, the composition is non-toxic to mammalian cells. In one aspect, the composition includes from about 0.5 μM to about 100 μM Zn(II), or about 0.5, 1, 5, 10, 25, 50, 75, or about 100 μM Zn(II), or a combination of any of the foregoing values, or a range encompassing any of the foregoing values, where any value can be the upper or lower endpoint of a range. In another aspect, the composition includes from about 1 μM to about 25 mM avobenzone, or about 1, 25, 50, 100, 250, 500, or 750 μM, or about 1, 5, 10, 15, 20, or about 25 mM avobenzone, or a combination of any of the foregoing values, or a range encompassing any of the foregoing values, where any value can be the upper or lower endpoint of a range. In one aspect, the composition can be a lotion and includes about 10 μM Zn(II) and about 25 mM avobenzone. In an aspect, the Zn(II) can be present as ZnSO₄ or another zinc salt. In some aspects, the method can be performed once, or can be performed every day for a period of at least one week. In some aspects, the method can be performed every 2-4 hours for at least a week; that is, can be performed from about 6 to about 12 times per day, or about 6, 7, 8, 9, 10, 11, or about 12 times per day, or a combination of any of the foregoing values, or a range encompassing any of the foregoing values.

In one aspect, disclosed herein is a composition including avobenzone and a transition metal salt, such as, for example, CuSO₄, ZnSO₄, ZnCl₂, zinc lactate, or any combination thereof. In a further aspect, the composition includes at least one carrier or excipient such as, for example, polyethylene glycol (PEG), water, an emulsifier, nanoparticles, a caged delivery system, or any combination thereof.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a transition metal,” “a bacterium,” or “a carrier,” include, but are not limited to, mixtures or combinations of two or more such transition metals, bacteria, or carriers, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a transition metal refers to an amount that is sufficient to achieve the desired improvement in the property modulated by the formulation component, e.g. achieving the desired level of antibacterial activity in the disclosed compositions. The specific level in terms of wt % in a composition required as an effective amount will depend upon a variety of factors including the amount and type of inactive ingredients, location of infection, severity of infection, and the specific strain of bacterium causing the infection.

As used herein, the terms “optional” or “optionally” means that the subsequently described event or circumstance can or cannot occur, and that the description includes instances where said event or circumstance occurs and instances where it does not.

As used herein, “nanoparticles” refers to synthetic, semisynthetic, or natural polymeric particles having an average diameter typically ranging from about 10 to about 100 nm. In one aspect, nanoparticles can be biodegradable and/or biocompatible, and can include, but are not limited to, polyacrylamide, polyacrylate, chitosan, polylactic acid, polyglycolic acid, combinations thereof, copolymers thereof, and derivatives thereof. Alternatively, in some aspects, nanoparticles can be inorganic, such as, for example, inert metals (e.g. Au, Ti) or metal oxides (e.g., iron oxide) that can form spheres at the nanometer scale. In any of these aspects, the nanoparticles can be surface functionalized and/or be present in a core-shell configuration, where the shell is the same as or different from the core. In one aspect, a nanoparticle has a large surface area to volume ratio, which may be useful for associating with drugs. Alternatively, the drugs can be encapsulated in an inner layer of the nanoparticles. In one aspect, nanoparticle structures and properties can facilitate uptake of the associated or included drugs by cells.

As used herein, a “caged delivery system” refers to a hollow micro- or nano-scale drug delivery system wherein one or more active ingredients are held inside the cage (e.g., a protein or polymeric molecule that surrounds the one or more active ingredients). In some aspects, the cages can be self-assembling protein subunits, other polymeric systems, or the like, and cages can be made of the same protein/polymer or several different proteins and/or polymers. In some aspects, caged delivery systems can include viral proteins, ferritin-related proteins, heat shock proteins, virus like particles, and the like. In some aspects, the external faces of caged systems can include attachment points for active ingredients, targeting moieties, and the like. In some aspects, the opening of the caged delivery system can be triggered by a change in environment (e.g., pH, temperature, salt concentrations, osmotic shock, or the like), allowing the cage to release its contents.

Unless otherwise specified, temperatures referred to herein are based on atmospheric pressure (i.e. one atmosphere).

### Pharmaceutical Compositions

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom, or condition thereof, such as an antibiotic resistant bacterial infection. The effect can be therapeutic in terms of a partial or complete cure of a disease, condition, symptom, or adverse effect attributed to the disease, disorder, or condition. The term “treatment” as used herein can include any treatment of antibiotic resistant bacterial infection in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder, or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound (including, but not limited to, zinc, avobenzone, and combinations thereof) or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound (including, but not limited to, zinc, avobenzone, and combinations thereof) and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of the compound(s) and/or pharmaceutical composition, by changing the compound(s) and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “contacting” as used herein refers to bringing a compound (including, but not limited to, zinc, avobenzone, and combinations thereof) or pharmaceutical composition in proximity to a cell, a target protein, or other biological entity together in such a manner that the compound(s) or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

In various aspects, the present disclosure relates to pharmaceutical compositions comprising a therapeutically effective amount of at least zinc, avobenzone, or a combination thereof, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In various aspects, the present disclosure also relates to a pharmaceutical composition comprising a pharmaceutically acceptable carrier or diluent and, as active ingredient, a therapeutically effective amount of a compound (including, but not limited to, zinc, avobenzone, and combinations thereof), a pharmaceutically acceptable salt, a hydrate thereof, a solvate thereof, a polymorph thereof, or a stereochemically isomeric form thereof. In a further aspect, a disclosed compound, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

The pharmaceutical compositions disclosed herein comprise a transition metal, avobenzone, and/or a combinations thereof as active ingredients, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds (i.e., zinc, avobenzone, and the like) described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The composition may be administered as a dosage that has a known quantity of the zinc and avobenzone.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus, and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a non-aerosol spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency. In another example, the cream or ointment can be further diluted with one or more carriers or excipients to achieve the desired active ingredient concentration.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin, and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier, and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic, or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin, and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets, or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs, and solutions; while carriers such as starches, sugars, microcrystalline cellulose, diluents, granulating agents, lubricants, binders, disintegrating agents, and the like can be used to form oral solid preparations such as powders, capsules, and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate, and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate, and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants, and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions, or suspensions, including saline and buffered media. Parenteral vehicles can include mannitol, normal serum albumin, sodium chloride solution, Ringer's dextrose, dextrose and sodium chloride, lactated Ringer's, and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

In one aspect, the amounts of transition metal salt and avobenzone can vary based on any solvents, carriers, and/or excipients included in the disclosed pharmaceutical compositions. In one aspect, avobenzone has limited solubility in water but higher solubility in fatty compositions (e.g. creams and lotions). Thus, in one aspect, achievable concentrations of avobenzone may be higher in topical lotions and creams and may be lower in aqueous compositions for dispensing as sprays, injections, IV solutions, and the like. In another aspect, under certain conditions where avobenzone solubility is low, including an increased amount of zinc in the compositions results in stable aqueous solubility and retention of antibacterial properties.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered aspects, which should not be confused with the claims.

Aspect 1. A method for treating or preventing a bacterial infection in a subject, the method comprising administering a composition comprising a transition metal and avobenzone to the subject.

Aspect 2. The method of aspect 1, wherein the transition metal comprises copper, zinc, manganese, nickel, cobalt, silver, iron, another transition metal, or any combination thereof.

Aspect 3. The method of aspect 1 or 2, wherein the transition metal is in a +1 or +2 oxidation state.

Aspect 4. The method of any one of aspects 1-3, wherein the transition metal comprises Zn(II).

Aspect 5. The method of any one of aspects 1-4, wherein the subject is a mammal, bird, reptile, or amphibian.

Aspect 6. The method of aspect 5, wherein the subject is a human, mouse, rat, guinea pig, rabbit, dog, cat, horse, cattle, swine, goat, sheep, chicken, turkey, duck, or another pet, research animal, or livestock animal.

Aspect 7. The method of aspect 5, wherein the mammal is a human.

Aspect 8. The method of any one of aspects 1-7, wherein the composition is administered topically, orally, intravenously, or by injection.

Aspect 9. The method of any one of aspects 1-8, wherein the composition is administered topically and is formulated as a cream, an ointment, a paste, a gel, a lotion, a milk, a suspension, an aerosol, a non-aerosol spray, a foam, a dusting powder, a pad, a patch, or any combination thereof.

Aspect 10. The method of aspect 8 or 9, wherein the composition is administered to a site comprising the skin of a subject, a wound, a burn, a surgical incision, or any combination thereof.

Aspect 11. The method of aspect 8, wherein the composition is administered orally and is formulated as tablets, capsules, pills, powder, granules, a suspension, a syrup, an emulsion, or any combination thereof.

Aspect 12. The method of any one of aspects 1-11, wherein the bacterial infection is caused by a Gram-positive bacterium.

Aspect 13. The method of aspect 12, wherein the Gram-positive bacterium is methicillin-resistant Staphylococcus aureus (MRSA).

Aspect 14. The method of aspect 12 or 13, wherein the Gram-positive bacterium is resistant to bacitracin.

Aspect 15. The method of any one of aspects 1-14, further comprising administering an antibiotic to the subject.

Aspect 16. The method of aspect 15, wherein the antibiotic comprises amoxicillin, clavulanic acid, ampicillin, sulfobactam, penicillin, vancomycin, ceftriaxone, daptomycin, ciprofloxacin, levofloxacin, ofloxacin, clindamycin, erythromycin, gentamicin, tetracycline, sulfamethoxazole, trimethoprim, another antibiotic, or any combination thereof.

Aspect 17. The method of aspect 15 or 16, wherein the antibiotic and the composition are administered sequentially.

Aspect 18. The method of aspect 15 or 16, wherein the antibiotic and the composition are administered simultaneously.

Aspect 19. The method of any one of aspects 1-18, wherein the composition is non-toxic to mammalian cells.

Aspect 20. The method of any one of aspects 1-19, wherein the composition comprises from about 0.5 μM to about 100 μM Zn(II).

Aspect 21. The method of any one of aspects 1-20, wherein the composition comprises from about 1 μM to about 25 mM avobenzone.

Aspect 22. The method of aspect 20 or 21, wherein the composition comprises about 10 μM Zn(II) and about 25 mM avobenzone.

Aspect 23. The method of any one of aspects 1-22, wherein the method is performed once.

Aspect 24. The method of any one of aspects 1-22, wherein the method is performed from about 6 to about 12 times per day for a period of at least one week.

Aspect 25. A composition comprising avobenzone and a transition metal salt.

Aspect 26. The composition of aspect 25, wherein the transition metal salt comprises CuSO₄, ZnSO₄, ZnCl₂, zinc lactate, or a combination thereof.

Aspect 27. The composition of aspect 26, wherein the composition comprises from about 0.5 μM to about 100 μM ZnSO₄.

Aspect 28. The composition of aspect 26 or 27, wherein the composition comprises from about 1 μM to about 25 mM avobenzone.

Aspect 29. The composition of any one of aspects 26-28, wherein the composition comprises about 10 μM ZnSO₄ and about 25 mM avobenzone.

Aspect 30. The composition of any one of aspects 25-29, further comprising at least one carrier or excipient.

Aspect 31. The composition of aspect 30, wherein the at least one carrier or excipient comprises polyethylene glycol (PEG), water, an emulsifier, nanoparticles, a caged-delivery system, or any combination thereof.

Aspect 32. The composition of any one of aspects 25-31, wherein the composition is formulated as a cream, an ointment, a paste, a gel, a lotion, a milk, a suspension, an aerosol, a non-aerosol spray, a foam, a dusting powder, a pad, a patch, tablets, capsules, pills, powder, granules, a suspension, a syrup, an emulsion, or any combination thereof.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Methods

### Bacterial Strains and Culture Conditions

All S. aureus strains utilized in this study were routinely cultured in Mueller-Hinton medium at 37° C. shaking at 180 rpm prior to transfer into experiment-specific media. Except for the transposon mutants and clinical isolates, all strains used in this study were purchased from ATCC. Transposon mutants in the JE2 genetic background were obtained from BEI Resources and were subsequently cultured under the aforementioned conditions plus 5 μg/mL erythromycin to maintain the transposon insertion. The epidemic USA300 strain LAC was transformed with the plasmid pCM12 encoding GFP to generate the strain LAC-GFP, which was subsequently cultured in MH medium with 100 μg/mL spectinomycin to maintain the plasmid. Lastly, clinical multi-drug resistant MRSA strains were isolated, characterized for clinical drug resistance, de-identified, and obtained from UAB Laboratory Medicine. For the mouse experiments, USA300-LAC was cultured in Luria-Bertani (LB; Sigma-Aldrich) broth.

### Antibiotics, Compounds, and Reagents

The bioactive compound screening library was purchased from Selleckchem. Avobenzone was purchased from Selleckchem or Sigma. All compounds were reconstituted to 10 mM concentration in 100% anhydrous DMSO, aliquoted, and stored at −80° C. for long-term storage such that individual aliquots did not lose potency due to freeze-thaw cycles. Antibiotics were dissolved in ddH₂O, sterile filtered with 0.2 μm nylon filters, and stored as 10 mg/mL aliquots at −80° C. except for erythromycin. Erythromycin was dissolved in 200 proof ethanol to 20 mg/mL and stored as aliquots at −20° C. Copper sulfate and zinc sulfate heptahydrate salts were purchased from Sigma. Metal stock solutions were stored as 100 mM aliquots in ddH₂O at 4° C. following sterile filtration with 0.2 μm nylon filters. The metabolic indicator dye resazurin (Sigma) was stored at 800 μg/mL in ddH₂O at 4° C. following sterile vacuum filtration with a 0.2 μm nylon filter.

### Zn(II)-Dependent Antibiotic Screening and Analysis.

Zn(II)-dependent screening of the 1,654-compound Bioactive Screening Library (Selleckchem) was performed against USA300-LAC. The compound library was diluted to 800 μM in 100% anhydrous dimethyl sulfoxide (DMSO) in 96-well round-bottom plastic plates using a Beckman Coulter FXp robotic platform. The dilution plates were then covered with adhesive aluminum film and stored at −80° C. To reduce variability across screening days, high density USA300-LAC seed stocks were used to generate the inoculum for the screen. Briefly, USA300-LAC was cultured to mid-exponential phase in MH medium, washed twice with 1×RPMI 1640 to remove residual MH medium, and normalized to an OD₆₀₀ of 5 in 1×RPMI 1640+15% glycerol (supplier) for cryoprotection. The cells were aliquoted then stored at −80° C. for future use.

To perform the screen, a seed stock was thawed, briefly centrifuged to remove the medium, and normalized to an inoculum OD₆₀₀ of 0.01 in 1×RPMI 1640, allowing a 30-minute rest period in the medium. Dilution plates were thawed, and compounds were screened at 10 μM in 1×RPMI 1640 with and without either 25 μM CuSO₄ or 25 μM ZnSO₄ using the Beckman Coulter FXp robotic platform. Resazurin was added to the medium at a final concentration of 10 μg/mL as a measure of bacterial metabolism and surrogate marker of bacterial growth. All screening plates were incubated at 37° C., 5% CO₂ for 12 hours and metabolic conversion of resazurin to resorufin (ex. 530 nm, em. 590 nm) was determined using a Biotek Cytation 3 plate reader. Measurements were background subtracted and normalized as percent growth of the negative control wells on the same plate. Hit compounds were defined as compounds that reduced the growth of USA300-LAC by at least 50% relative to the vehicle-treated controls. Hit compounds were subdivided into metal-dependent, metal-inverse, or metal-independent by comparing the percent growth for a given compound without a metal to that of the same compound with a metal.

### Determination of Minimal Inhibitory Concentration and Drop Assays

The minimal inhibitory concentration (MIC) of the tested compounds was determined using dose-response curves as previously described. Briefly, each strain was cultured to mid-exponential phase in MH medium, as determined by OD₆₀₀ measurement. S. aureus cultures were then normalized to an OD₆₀₀ of 0.005 for transfer into the assay plates. Each strain was treated with compounds serially titrated in the assay medium in 96-well flat-bottom plastic plates with or without either CuSO₄ or ZnSO₄. Compound-untreated samples for each metal condition were used as negative controls. S. aureus were treated for 18-20 hours at 37° C. and 5% CO₂. Following the treatment period, bacterial growth was determined by measuring the absorbance of each well at 600 nm using a Biotek Cytation 3 plate reader. Measurements were then background adjusted and normalized to either the untreated control or the metal only control. The minimal inhibitory concentration (MIC) was defined as the minimal compound concentration that reduced the growth of the strain to 10% or less than that of the untreated control.

### Resistance Generation

Resistance studies were carried out over 30 days using S. aureus JE2 as the wild type parent strain, as this strain was used for the generation of the Nebraska transposon library that could be a most helpful tool for further target analysis. For the initial passage, JE2 was cultured to mid-exponential phase in MH broth and washed twice with 1×RPMI-1640 to remove residual MH medium. JE2 was transferred to 1 mL of each treatment made in 1×RPMI-1640 at a standard density of OD₆₀₀ of 0.01 (10 million cells/mL) and incubated at 37° C. with orbital shaking for 24 hours. Treatments were composed of 0.25×, 0.5×, 1×, 2×, and 4×MIC of avobenzone with 15 μM ZnSO₄ or the positive control rifampicin. Once per day, the previous passage would be visually assessed for growth, and the second highest concentration showing bacterial growth per treatment would be selected to inoculate the next passage at a 1:100 dilution. Individual colonies were then isolated from the selected culture by streak plating onto MH agar and tested for resistance to either rifampicin or avobenzone with 15 μM ZnSO₄ relative to the parent strain using a dose-response curve as described above.

### Binding Constant Determination

Binding constants were determined using UV/Vis-absorption spectroscopy by titrating AVB with Cu(I), Cu(II), and Zn(II). Briefly, 1 mM AVB in 100% anhydrous DMSO was titrated in HEPES (4-(2-hydroxyethyl)-1-piperazineethanesulfonic acid) buffer (pH=7.2-7.4) with 800 μM CuBr, CuSO₄, or ZnSO₄ at the following concentrations: 0 μM, 25 μM, 50 μM, 75 μM, 100 μM, and 150 μM. Once the gradient of solutions was prepared, full spectrum UV/Vis-absorption spectroscopy was performed to detect characteristic spectral changes that occur as a consequence of metal cation binding. Binding constants were calculated by importing the spectra of individual titrations with each metal into the UV 1:1 Bindfit program.

### Metal Complex Stoichiometry Determination

AVB, CuBr, and ZnCl₂ were dissolved in methanol (ACS grade) at 1 mM concentration. The binding stoichiometries of the AVB-Zn and AVB-Cu complexes were investigated by applying the method of continuous variation (often referred to as Job's method). Three replicates of a 10-solution series in 3.0 ml quartz cuvettes were prepared using the following ratios: (i) 3.0 ml AVB+0 ml metal, (ii) 2.7 ml AVB+0.3 ml metal, (iii) 2.4 ml AVB+0.6 ml metal, (iv) 2.1 ml AVB+0.9 ml metal, (v) 1.8 ml AVB+1.2 ml metal, (vi) 1.5 ml AVB+1.5 ml metal, (vii) 1.2 ml AVB+1.8 ml metal, (viii) 0.9 ml AVB+2.1 ml metal, (ix) 0.6 ml AVB+2.4 ml metal, and (x) 0.3 ml AVB+2.7 ml metal. The solutions were allowed to react at 298 K for 30 min. Following incubation, UV/Vis absorption at 580 nm was measured using a Cary 4000 UV/Vis spectrometer, with an empty quartz cuvette used as a standard.

### Eukaryotic Toxicity Assays

THP-1 cells and Jurkat T cells were originally purchased from the ATCC and cultured at 37° C., 5% CO₂ in 1×RPMI 1640 supplemented with 10% heat-inactivated fetal bovine serum, 100 U/mL penicillin, 100 μg/mL streptomycin, and 2 mM L-glutamine. Peripheral blood mononuclear cells (PBMCs) were isolated from commercially obtained buffy coats. Cell numbers and viability in the presence or absence of drug or metals were assessed using a Guava EasyCyte flow cytometer. This flow cytometer uses a capillary-based analytical system combined with a precision pump and provides precise absolute cell counts without the need of using reference beads. Each cell line was tested against serial titrations of a given compound starting at 30 μM to 0.04 μM with and without 15 μM ZnSO₄ or CuSO₄. Cells were seeded at a standard density of 100,000 cells/well in 96-well flat-bottom plastic plates at 37° C.+5% CO₂ for 24 hours then assessed for viability using forward and side scatter gating (FSC/SSC).

### Lotion Preparation and Modified Kirby-Bauer Diffusion Assays

Antibiotic lotions were formulated by using a commercially available polyethylene glycol (PEG)-based lotion (CeraVe) as a base. To ensure thorough mixing of the lotion and additives, the lotion was mixed with 30% (v/v) Tween-80 in sterile double distilled water (ddH₂O) to form an inert base lotion composed of lotion+3% (v/v) Tween-80. Zn-containing lotions were made by mixing this base lotion with 1.5 M ZnSO₄ dissolved in sterile ddH₂O for a final concentration of 10 mM ZnSO₄. An equal volume of sterile ddH₂O was added to lotions lacking Zn. Avobenzone (Sigma) or bacitracin (Sigma) dissolved in 100% DMSO were added to the control and Zn-containing lotions for a final concentration of 10 mM. The vehicle lotion was generated by adding an equivalent volume of 100% DMSO and sterile ddH₂O as used in the compound and Zn-containing lotions respectively. Each component was thoroughly incorporated into the lotion by vortexing for one minute, and each lotion was stored at 4° C. in the dark for up to two weeks. The antibacterial activity of each lotion was assessed using a modified Kirby-Bauer disc diffusion assay. MH agar plates with identical indentations in the agar were inoculated with 2 million cells/cm² of USA300-LAC. Lotions were dispensed into each indentation using a standard volume of 100 μL using sterile Luer lock syringes and 16-gauge needles. The plates were inverted and incubated for 18 hours at 37° C. The plates were then scanned and the zones of inhibition were measured and compared to that of the vehicle control for each treatment.

### Murine Wound Model

For use in in vivo wound experiments, methicillin-resistant S. aureus (MRSA) strain USA300 was grown overnight in Luria-Bertani (LB) broth (Sigma-Aldrich, St. Louis, MO) at 37° C. with 250 rpm shaking. Bacteria were washed and resuspended to 10⁴ CFUs/mL in sterile phosphate-buffered saline (PBS; ThermoFisher Scientific, Waltham, MA, USA). Commercially available sterile gauze was trimmed to 6 mm square dressings and incubated with bacterial culture at room temperature for 10 min prior to use.

Mouse surgical methods were adapted from the wound model of Brandenburg, et al. On study day 0, 5-6 month old mice were anaesthetized via intraperitoneal injection with 85.5 mg/kg ketamine hydrochloride (Vedco Inc., St. Joseph, MO) and 12.5 mg/kg xylazine (MWI, Boise, ID) in cocktail. The cranial thoracodorsal region was shaved and prepared for surgery using 70% EtOH (ThermoFisher Scientific, Waltham, MA, USA) and topical analgesic (Burn Jel; Water-Jel Technologies LLC, Carlstadt, NJ) for pain control. A silicon O-ring (McMaster-Carr, Douglasville, GA) was attached to the skin via four to six 5-0 interrupted nylon sutures and secured with tissue adhesive (GLUture; Zoetis Inc., Kalamazoo, MI). A 6 mm diameter wound was created within the ring using a biopsy punch. Bacterial-laden dressings were applied to the wound, allowed to incubate, and removed four hours later.

Each mouse was housed individually and monitored following the procedure until fully ambulatory. On day 1 post-infection, treatment was initiated with either 100 μL of either vehicle control lotion, avobenzone lotion, AVB-Zn lotion, or commercially-available bacitracin ointment (Bacitraycin Plus; First Aid Research Corp., Jupiter, FL) applied to the wound surface via sterile syringe. Treatments were applied daily through study day 7, <2 hours prior to the start of the facility dark cycle. On study day 8, mice were euthanized via intraperitoneal injection of 200 μL pentobarbital sodium (390 mg/mL; Vortech, Dearborn, MI). Throughout the study, mice were monitored for signs of distress (severe weight loss, prolonged lethargy), and euthanized when necessary under the advisory of UAB veterinary staff. All animal experiments at UAB were conducted in accordance with UAB Institutional Animal Care and Use Committee (IACUC) approved protocols. All animal experiments used wild-type C57BL/6 mice (The Jackson Laboratory, Bar Harbor, ME) 5-6 months of age. Animals were bred and housed in standard cages with a 12 hour light/dark cycle at 71-75° F. with ad libitum access to food and water. Experimental groups were composed of even numbers of males and females.

Mouse survival was assessed on each day during the experiment. Significance in mouse survival between groups was determined using a log rank test for trend. On day 8 of the study, swabs of the wounds of the sacrificed mice were performed with sterile cotton swabs. CFUs were enumerated by inoculating mannitol salt agar plates with the swabs and incubating the plates overnight at 37° C. Mean CFUs were calculated for each treatment group, and significance was determined using a Kruskal-Wallis test.

### Example 2: Results

### Identification of Zn-Activated Antibiotics Against MRSA

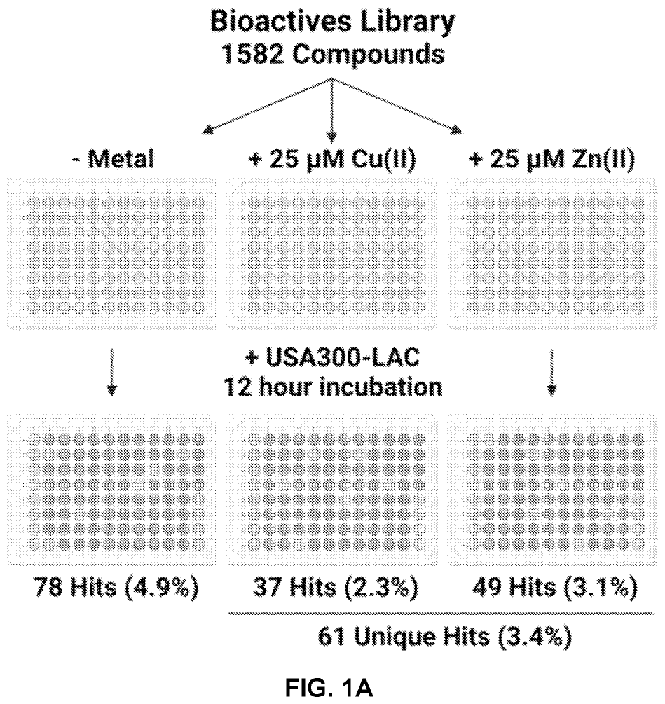

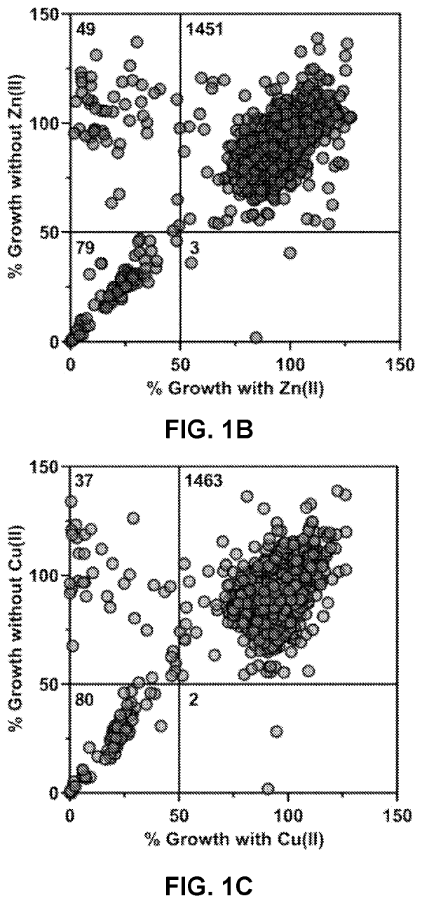

To determine whether Zn, as the only transition metal without redox activity, has the capacity to activate otherwise inactive compounds to exert antibacterial activity, a library of 1,654 FDA-approved drugs and compounds with known bioactivities were screened in the presence or absence of Zn. The screen was performed against the well described MRSA strain USA300-LAC in chemically-defined RPMI-1640 medium. Given that copper-complexed compounds are the primary focus of current antibacterial metallodrug research, a parallel reference screen was performed using copper (Cu) as the activating transition metal to directly compare the efficacy of Zn and Cu to promote antibacterial activation of compounds. Each compound was tested for antibacterial activity without metal, with 25 μM ZnSO₄, or with 25 μM CuSO₄. Growth after overnight incubation at 37° C. was determined by measuring changes to the fluorescence of the metabolic indicator dye resazurin (FIG. 1A). Compounds that reduced the growth of USA300-LAC by 50% were classified as hits.

The screen identified 143 hits out of a total 1582 compounds for a hit rate of approximately 9%. Of the compounds identified, 78 compounds (4.9%) were classified as independent and subsequently inhibited staphylococcal growth independent of a metal (FIGS. 1B-1C; lower left quadrant). As expected, this category included antibiotics known to target S. aureus (42), such as the rifamycin class and antiseptics (8), while narrow spectrum antibiotics targeting other organisms were exclusively classified as inactive. Importantly, bacitracin, a common cyclic polypeptide antibiotic used in first-aid ointments that requires Zn for its activity, was accurately identified as Zn-dependent. This highlights both the accuracy of Zn-dependent screening and the need to include metals as antibiotic screening conditions to recognize successful antibiotic molecules whose activities would otherwise be overlooked. Screening with physiologically relevant concentrations of both Zn and Cu nearly doubled the hit rate of the screen as a whole. Zn activation was displayed in 49 (3.1%) of the otherwise inert compounds (FIG. 1B), and antibacterial Cu activation was seen for 37 of the compounds (2.3%) (FIG. 1C). While some compounds were activated by both Cu and Zn (25), compounds solely activated by Zn (24) within the screen predominated over compounds with Cu-specific activation (12). Zn was thus an efficient activating component for metallo-antibiotics. This would suggest the inherent redox potential of a metal like Cu is not necessarily required to induce the metallo-antibiotic activity of small chemical molecules, thereby broadening the possible modes of action a metallo-antibiotic could possess. Zn-activated compounds could act as Zn ionophores and cause mismetallation of essential enzymes by the intracellular release of Zn, as has been previously described. Alternatively, compounds could directly coordinate with Zn, thereby altering the compound conformation to a form active against a specific target, or Zn-activated molecules may simply possess increased membrane permeability. Regardless of the mechanism, it is evident that consideration of physiological Zn concentrations during the drug screening process can expand the chemical space of discoverable active compounds.

### Avobenzone is a Potent Zn-Activated Anti-MRSA Metallo-Antibiotic

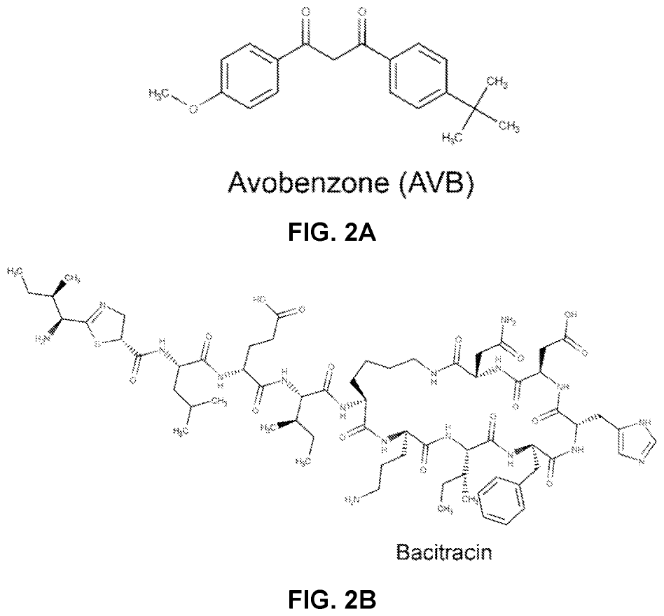

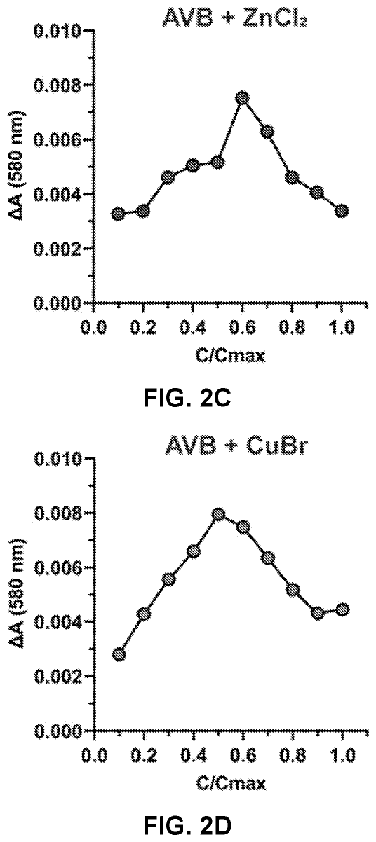

While the drug screen identified a series of FDA-approved drugs as Zn-activated compounds against MRSA, most are known to have serious side effects, as they belong to the classes of cancer agents (e.g. ponatinib, tamoxifen, toremifene citrate) or antifungals (e.g. clotrimazole, bifonazole, tioconazole), and would thus may not warrant further exploration. An exception to this is avobenzone, which was identified as being activated by both Zn and Cu. Avobenzone (AVB), or 1-(4-tert-butylphenyl)-3-(4-methoxyphenyl)propane-1,3-dione, is an FDA-approved UV-A filter and a common active ingredient in commercial sunscreens and beauty products. It is composed of a central 1,3-diketone group connecting two 2,4-substituted phenyl rings with a methoxy and a tert-butyl substituent (FIG. 2A).

Given that ketone groups are known to coordinate with transition metals, it was hypothesized AVB would form a complex with Zn to inhibit S. aureus. It was first confirmed that AVB complexed with Cu and Zn (Table 1). UV/vis spectroscopy binding studies demonstrated that Zn(II) (K_{D}=5.9 μM) and Cu(II) (K_{D}=4.4 μM), but not Cu(I), efficiently bound to AVB, confirming the existence of an AVB-Zn and AVB-Cu complex specific to their divalent form. While these constants fall short of the binding activity of high-affinity metal chelators, such as TPEN (N,N,N′,N′-tetrakis(2-pyridinylmethyl)-1,2-ethanediamine) or EDTA (2,2′,2″,2′″-(Ethane-1,2-diyldinitrilo)tetraacetic acid), for which K_{D}s are reported in the picomolar to femtomolar range, the Kos are similar to those determined for bacitracin. Bacitracin also had similar affinities for Zn(II) (K_{D}=5.7 μM) and Cu(II) (K_{D}=1.4 μM) as AVB. Thus, AVB has a similar affinity for Zn as a Zn-binding, FDA-approved antibiotic.

TABLE 1
Metal Binding Kinetics of AVB and Bacitracin

| | |
|---|---|
| Avobenzone | Bacitracin |

| Metal | K_{A} (M⁻¹) | K_{D} (μM) | K_{A} (M⁻¹) | K_{D} (μM) |
|---|---|---|---|---|
| Cu(I) | 6900 | 114.93 | 9299 | 107.54 |
| Cu(II) | 225,279 | 4.43 | 738,233 | 1.35 |
| Zn(II) | 168,982 | 5.92 | 172,757 | 5.79 |

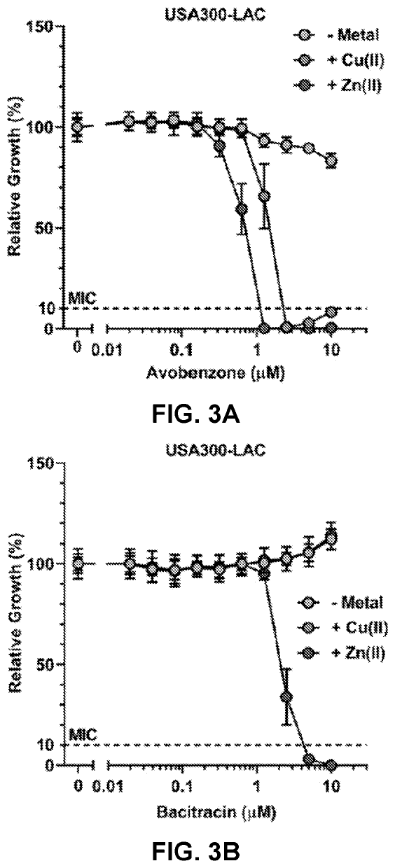

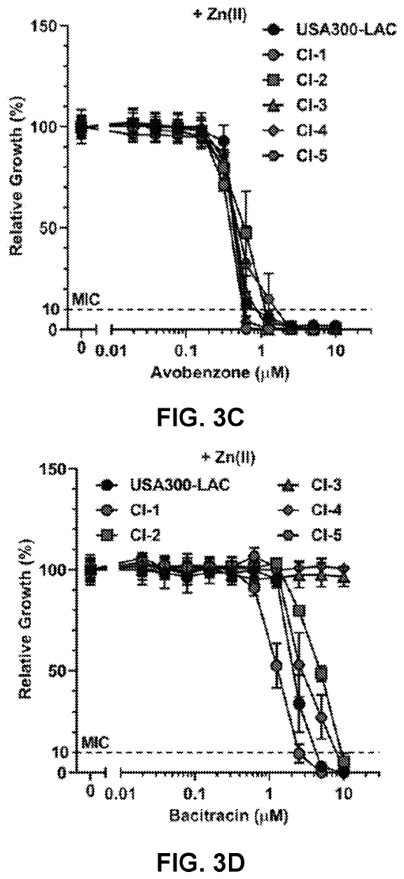

Having demonstrated AVB complexes with divalent Zn and Cu, the minimal inhibitory concentration (MIC), or the lowest concentration causing 90% inhibition of bacterial growth, was next determined for AVB against MRSA. For this purpose, challenge assays of AVB in the presence and absence of Zn or Cu were performed against USA300-LAC, and inhibition was determined by normalizing A600 measurements, a marker of bacterial growth, to that of the compound-untreated control of each metal condition. In line with the screening results for AVB, AVB alone exerted no antibacterial activity against USA300-LAC but gained antibacterial activity in the presence of a metal, with AVB-Cu effective at inhibiting the growth of USA300-LAC at 2.5 μM (FIG. 3A). However, AVB-Zn was the most potent of these combinations, with an MIC of 1.25 μM against USA300-LAC, indicating that large parts of the antibacterial effect exerted by AVB-Zn under these experimental conditions were bactericidal. In contrast, the MIC of bacitracin-Zn (5 μM) was four-fold higher than that of AVB-Zn (FIG. 3B), underscoring the potential of AVB-Zn as an antibiotic treatment.

It was next tested whether clinical multidrug-resistant MRSA isolates possessed pre-existing resistance mechanisms against AVB-Zn (FIG. 3C). None of the five tested isolates showed any signs of pre-existing resistance to AVB-Zn relative to USA300-LAC, with the MICs ranging from 0.6-2.5 μM depending on the isolate. While this panel only includes a limited number of strains, the resistance profiles of these strains cover most major antibiotic classes in clinical use, implying AVB-Zn has an alternative mechanism of action than most antibiotics currently in use (Table 2).

This finding was particularly interesting given that four out of five of these clinical isolates possessed resistance to bacitracin-Zn. CI-4 and CI-5 (MIC=10 μM) were partially resistant to bacitracin-Zn relative to USA300-LAC, while CI-2 and CI-3 were fully resistant to the complex in the disclosed system (FIG. 3D). While resistance to Zn-activated compounds can develop, these findings suggest that these resistance mechanisms do not necessarily provide cross-resistance to other Zn-activated compounds, further indicating Zn activation does not induce a single mode of action.

Cu-activated avobenzone (AVB-Cu) was somewhat less potent. In solution, AVB-Cu started to be inhibitory at 1.25 μM, but did not reach IC₉₀ until 10 μM, but according to drop assays, was bactericidal starting at 2.5 μM.

It was next tested whether clinical multidrug-resistant MRSA isolates may already have pre-existing resistance mechanisms against AVB-Zn. None of the tested five isolates showed any signs of pre-existing resistance, with the MICs of AVB-Zn ranging from 0.6-1.25 μM depending on the isolate. While this panel only includes a limited number of strains, the resistance profiles of these strains cover almost most major antibiotic classes in clinical use, which would imply that AVB-Zn acts in novel manner (Table 2).

TABLE 2
Resistance Profiles of Multi-Drug Resistant MRSA Isolates

| Drug Target | Antibiotic Class | Antibiotic Formulation | CI-1 | CI-2 | CI-3 | CI-4 | CI-5 |
|---|---|---|---|---|---|---|---|
| Peptidoglycan | β-lactam | Amoxicillin + clavulanic | R | R | R | R | R |
| synthesis | | acid | | | | | |
| | | Ampicillin + sulfobactam | R | R | R | R | R |
| | | Ampicillin | R | R | R | R | R |
| | | Penicillin | R | R | R | R | R |
| | Glycopeptide | Vancomycin | S | S | S | S | S |
| | Cephalosporin | Ceftriaxone | R | R | R | R | R |
| Cell membrane | Cyclic lipopeptide | Daptomycin | S | S | S | S | S |
| DNA gyrase | Fluoroquinolone | Ciprofloxacin | S | R | S | S | R |
| | | Levofloxacin | S | R | S | S | R |
| | | Ofloxacin | R | R | R | R | R |
| Translation - | Lincomycin | Clindamycin | R | R | S | S | R |
| 50S subunit | | | | | | | |
| | Macrolide | Erythromycin | R | R | R | R | R |
| Translation - | Aminoglycoside | Gentamicin | S | S | S | S | S |
| 30S subunit | | | | | | | |
| | Tetracycline | Tetracycline | R | S | S | S | S |
| Folic acid | Sulfonamide | Sulfamethoxazole + | S | S | S | S | S |
| metabolism | | trimethoprim | | | | | |

The MICs of AVB-Cu ranged from 2.5-10 μM for the tested isolates. Of note, the difference in MICs for AVB-Cu can be associated with the presence or absence of the described Cu export genes, copZ and copX, in these isolates. Strains lacking these genes were more sensitive to AVB-Cu, suggesting AVB-Cu may act as a Cu ionophore. The presence of Cu transporters did not affect AVB-Zn activity.

The finding that none of the clinical isolates had any pre-existing AVB-Zn resistance was particularly interesting given the finding that AVB-Zn was also active against bacitracin resistant clinical isolates. Bacitracin is an FDA-approved antibiotic used in first-aid ointments that inhibits the formation of a peptidoglycan precursor only when complexed with Zn. Bacitracin was found to be completely inactive against USA300-LAC in the disclosed system in the absence of Zn and could not be activated by Cu. While bacitracin in its Zn-activated form had an MIC of 2 μM against USA300-LAC and two clinical isolates (CI-1320 and CI-1328), two of the clinical isolates (CI-1323 and CI-1324) were resistant to bacitracin-Zn at all concentrations tested, and one strain (CI-1321) was partially resistance. These findings suggest that while resistance against Zn-activated compounds can develop, these resistance mechanisms do not necessarily provide cross-resistance to other Zn-activated compounds.

### Optimal AvoZ Concentrations

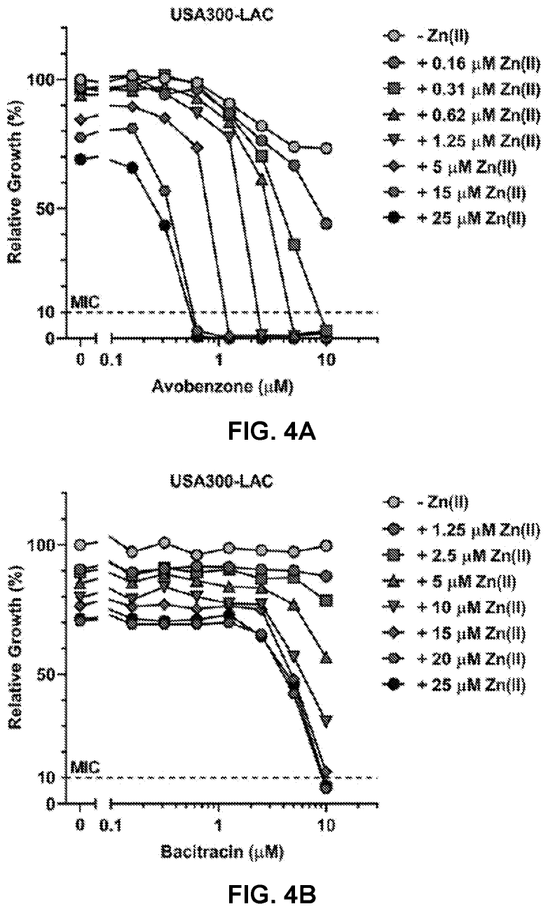

Drug screening conditions are usually designed to maximize hit discovery and in this case were performed at the higher end of physiological relevant Zn concentrations. While a screening concentration of 25 μM of zinc may be physiologically relevant under some conditions (e.g. inside the phagosome of macrophages), it was also desirable to determine the lowest effective concentrations at which a combination of Zn and avobenzone would provide full AVB-Zn activity. Dose matrix experiments were thus performed, titrating zinc against increasing avobenzone concentrations (FIG. 4A). These data indicated that the full activity of AVB-Zn was reached by combining 2.5 μM avobenzone with 2.5 μM of Zn. Higher concentrations of either Zn or avobenzone only resulted in a neglectable activity increase. As a reference and to determine the potency of AVB-Zn, the same dose matrix experiments were performed for bacitracin. Surprisingly, bacitracin exerted only minimal antibacterial effects at concentrations lower than 10 μM, and only reached the MIC once 10 μM bacitracin were combined with 10 μM zinc (FIG. 4B). These data would suggest that at a 10 μM zinc concentration AVB-Zn is >15-fold more against potent MRSA USA300 than the FDA-approved zinc-salt antibiotic bacitracin.

### Eukaryotic Toxicity

It was next sought to determine the toxicity of AVB-Zn against eukaryotic cells in this system by performing challenge assays of AVB, AVB-Zn, and AVB-Cu against human Jurkat T cells and patient-derived PBMCs from three healthy donors. Cell viability and proliferation were measured after 24 h of treatment using flow cytometric analysis. Cell viability was defined as the per-centage of cells in a forward scatter/side scatter-based viability gate, and cell number was defined as the absolute number of cells within this gate. The latter analysis can detect inhibitory effects to cell proliferation that do not result in cell death and may be the most sensitive means to detect toxic drug effects.

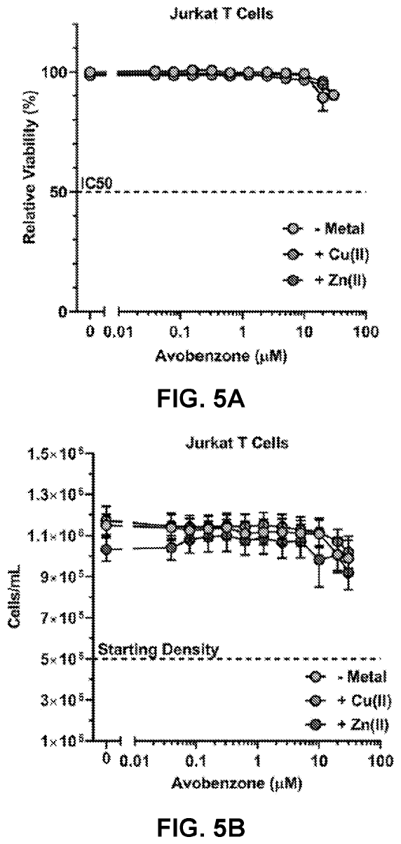

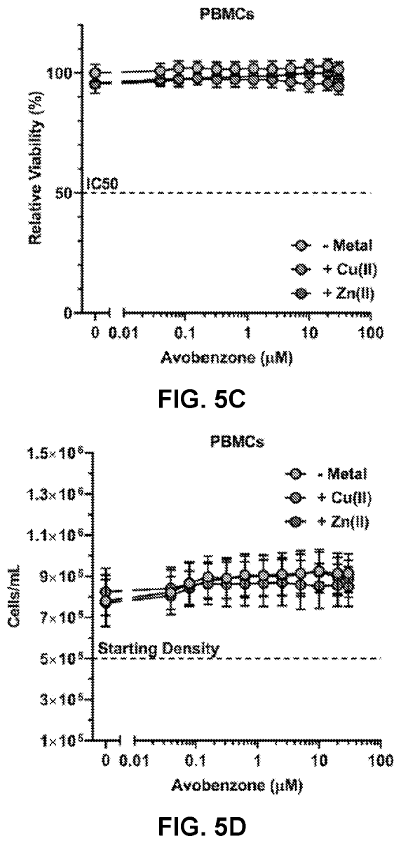

In contrast to the potent antibacterial activity that avobenzone exerted as a Zn-activated metallodrug, no relevant cytotoxicity of free or zinc-activated avobenzone was detected against human cells. AvoZ and avobenzone were initially titrated on human Jurkat T cells and the monocytic THP-1 cells (0.4-30 μM±15 μM Zn). After 24 hours cell viability (FIGS. 5A and 5C) and cell numbers (proliferation) (FIGS. 5B and 5D) were determined by flow cytometric analysis. Cell viability was defined as the percentage of cells in a forward scatter/side scatter-based viability gate, and cell number was defined as the absolute numbers of cells within this viability gate. The latter analysis can detect inhibitory effects to cell proliferation that do not result in cell death and may be the most sensitive means to detect toxic drug effects. In these experiments, minor toxicity was observed for AVB-Zn on Jurkat T cells at 30 μM AVB-Zn (FIGS. 5A-5B) and no effect of AVB-Zn on cell viability or cell count was apparent for THP-1 cells (FIGS. 5C-5D).

Testing metallo-drugs on tumor cell lines is convenient and commonly done, but it is also insufficient, as tumor cells have different metal ion requirements than primary T cells. In general, tumor cell lines are more dependent on the availability of metals such as zinc or copper, so the absence of toxicity exerted by a potent zinc and copper chelator on tumor cell lines was very promising. Nevertheless, it is essential to evaluate metallodrug toxicity on primary human cells. For this purpose, PBMCs from three individual donors were used that were either left untreated or exposed AVB-Zn. In these experiments, no loss of viability and no effect on cell numbers were observed even at 30 μM AVB-Zn. Given that the MIC of AVB-Zn for MRSA was determined to be ˜1 μM, these data suggest a therapeutic index for the Zn-avobenzone complex that is >30.

### Resistance Generation

Given that pre-existing resistance to AVB-Zn was not observed in clinical MRSA isolates, it was next determined whether it was possible to generate bacterial resistance to AVB-Zn in vitro. This would provide valuable information on the clinical value of AVB-Zn. For this effort, the MRSA strain JE2 was used; this is a modified version of USA300-LAC cured of all plasmids and the parent strain for the S. aureus Nebraska Transposon Library. This strain would allow use of the transposon library for future target analysis. JE2 showed sensitivity to AVB-Zn that was comparable to that of USA300-LAC.

To generate AVB-Zn resistance, JE2 was cultured over a range of avobenzone concentrations (0.25-4×the MIC) with 15 μM Zn. After overnight incubation at 37° C., the culture with the second highest AVB-Zn concentration containing bacterial growth was passaged 1:100 into fresh treatment cultures with the same range of AVB-Zn concentrations. As a control, the same method was used to generate rifampicin resistant JE2 mutants, rifampicin being an antibiotic known for its ability to rapidly generate resistance when used as a monotherapy. Following this experimental design, bacteria were passaged for a total of 30 days to generate resistance. Bacterial clones from cultures that by visual assessment had developed resistance to AVB-Zn or rifampicin were isolated by streak plating onto MH agar.

As expected, rifampicin resistance developed rapidly. Rifampicin-resistant clones could be isolated beginning on day 5. Interestingly, in cultures that were initiated using sub-MIC concentrations of AVB-Zn apparent AVB-Zn resistance developed within three days when culture growth was assessed visually. However, when the isolated clones were assessed for stable resistance development, even after repeating the step pressure method for 30 days, the clones were no more resistant to AVB-Zn than the parent strain. This would imply that MRSA is capable of adapting to AVB-Zn if given initial sub-inhibitory concentrations, but does not generate stable, genomic resistance mutations. The inability of MRSA to develop AVB-Zn resistance should bode well for possible clinical translation, however, it poses a problem for the identification of the actual bacterial target of AVB-Zn, which is commonly and most efficiently done by the identification of the genetic changes that enable resistance. The most likely explanation for the inability of USA300-LAC to develop mutation-based resistance is that AVB-Zn generally targets a series of Zn-dependent proteins. In this case, a single mutation would not be sufficient to escape the antibacterial effect of AVB-Zn. Given that there is a relatively significant number of approved drugs for which the actual mechanism of action is unknown, and being presented with the opportunity to repurpose a sunscreen component with an exceptional safety record as an anti-MRSA drug, it was decided to focus on testing the ability of AVB-Zn to combat MRSA infection in a mouse model.

AVB-Zn Efficacy in a Wound Model of S. aureus Infection in Mice

As Staphylococcus aureus is known as an opportunistic pathogen that frequently occurs in non-healing skin wounds, an animal model was chosen that would replicate the phenotype of chronic, deep skin wounds to evaluate the possibility that avobenzone would also act as an antibacterial in vivo.

In a first step, it was demonstrated that it is possible to prepare antibacterial AVB-Zn lotions using a modified Kirby-Bauer disk diffusion susceptibility assay. Briefly, agar plates with 8 mm diameter indentations were produced, which were filled with the different lotion preparations after the plates were inoculated with USA300-LAC. Drug efficacy was determined by monitoring the zone of inhibition. Neither vehicle lotion nor avobenzone lotion (50 μM) nor Zinc-lotion (10 μM) produced a zone of inhibition, whereas AVB-Zn lotions inhibited growth of USA300-LAC, demonstrating that the antibacterial AVB-Zn can be achieved in lotion preparations.

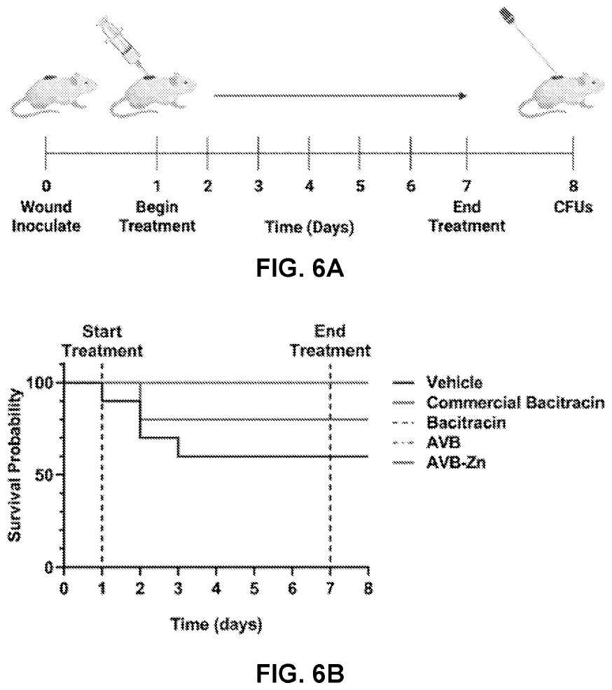

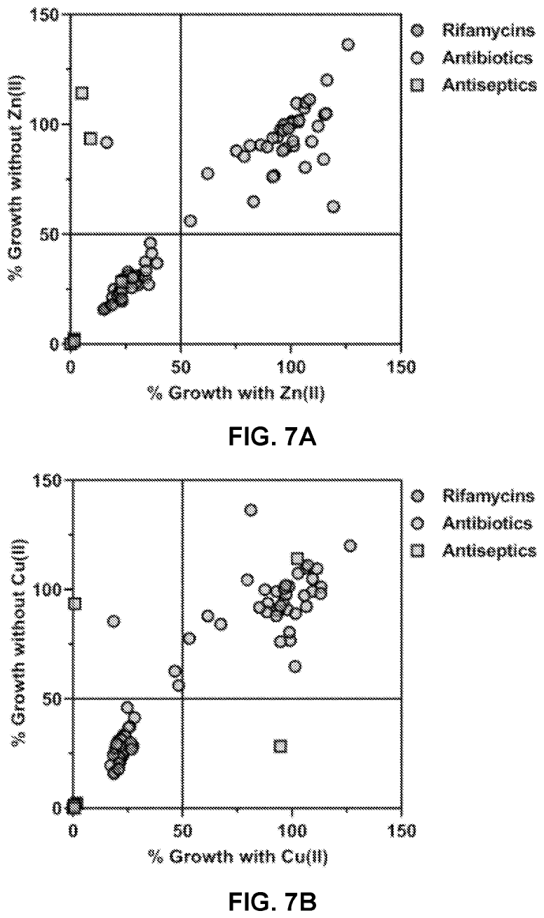

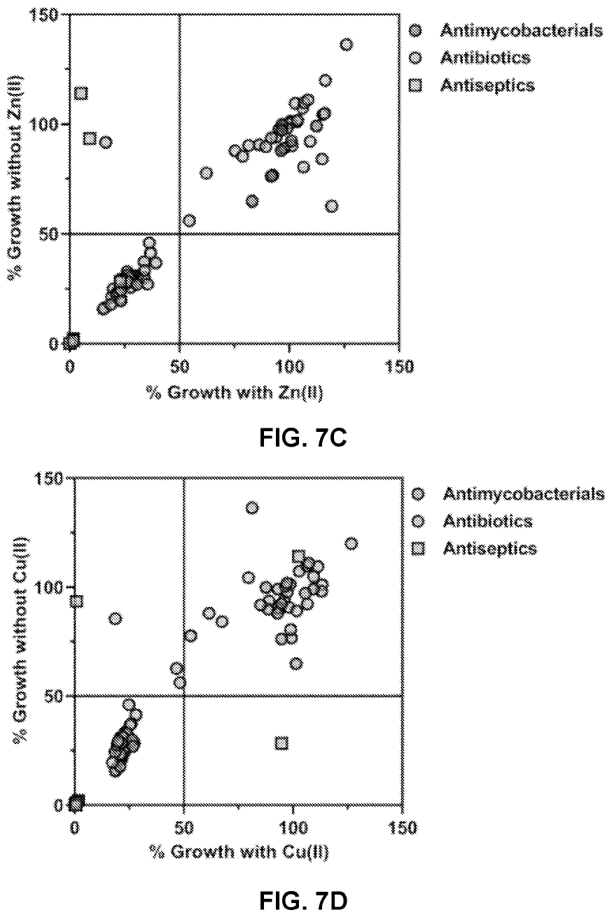

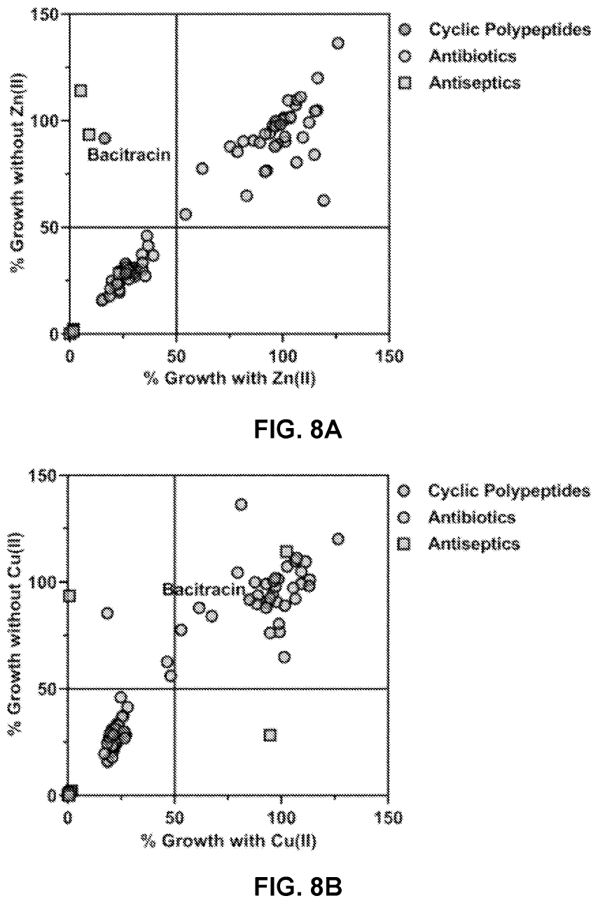

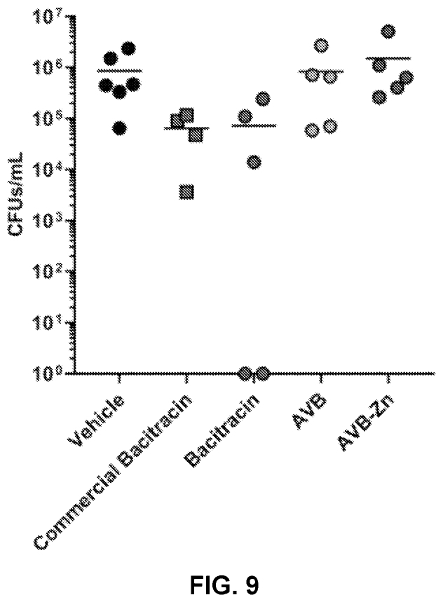

First, lotion based treatments of AVB-Zn against USA300-LAC were prepared and in vitro efficacy was tested using a modified Kirby-Bauer disc diffusion assay. Briefly, an inert base lotion composed of a commercial PEG-based lotion and 3% (v/v) Tween-80 was mixed with components of each treatment to make the various treatment lotions. MH agar plates with 100 μL volume indentations were inoculated with USA300-LAC, and the indentations were filled with each treatment lotion. Zones of inhibition were measured following overnight incubation. Using this method, the efficacy of four different lotion preparations was compared, a vehicle control, Zn alone, bacitracin-Zn, and AVB-Zn, to inhibit the growth of USA300-LAC. These results were compared to a commercially available bacitracin-Zn first-aid ointment. No inhibitory effect was observed against USA300-LAC with the vehicle control and Zn control lotions. However, both the bacitracin-Zn and AVB-Zn lotions generated zones of inhibition. Interestingly, the commercial bacitracin-Zn ointment did not noticeably inhibit USA300-LAC growth, indicating the size of the zones of inhibition are likely due to limitations in the rate of diffusion in this system. Once efficacious lotion treatments were established, the preparations were tested in an S. aureus wound infection mouse model to determine the in vivo efficacy of AVB-Zn. These results were also compared to the efficacy of the commercial bacitracin-Zn ointment. Briefly, the cranial thoracodorsal region of each C57BL/6 mouse was shaved and cleaned for surgery. A silicon O-ring was secured to the shaved area and a biopsy punch was used to create a 6 mm wound within each O-ring. A 6 mm dressing coated with USA300-LAC was then applied to the wound. The wound dressing model is a modification of a previously established model for the study of delayed wound healing caused by Pseudomonas aeruginosa biofilms. Given the dimension of the punch biopsy, the ensuing infection would be considered equivalent to a deep tissue infection. Treatment began 24 h post-application of the bacteria and was re-applied once a day for 1 wk. Mouse survival was monitored over the course of the study, and CFUs were enumerated from wound swabs upon sacrifice (FIG. 6A).

A significant difference (P=0.0173) in mouse survival was observed over the course of the experiment. Of the mice treated with just a vehicle control lotion (n=10), 4 mice (40%) died or were euthanized due to signs of insurmountable disease within the first 72 h (FIG. 6B). In the group of mice treated with the commercial bacitracin preparation (n=5) one mouse (20%) died after 48 h, while no mice from the AVB-Zn treatment group died (n=5). Interestingly, there was also no death observed in the group of mice treated with AVB lotion lacking Zn (n=5), which suggests AVB may locally coordinate with Zn and become activated to exert its antibacterial effect. Though additional studies, especially in systemic infection models, are required to optimize the AVB-Zn concentrations needed therapeutically, these in vivo data support the in vitro results that AVB-Zn protects against in-surmountable MRSA infection similarly to a known antibiotic. AVB-Zn did not significantly reduce tissue CFUs relative to the vehicle-treated control. While disappointing, this is not entirely surprising. Even under optimal in vitro conditions where AVB-Zn is solubilized, AVB-Zn does not cause full clearance of MRSA cultures under any of the concentrations tested in the challenge assays, suggesting AVB-Zn is bacteriostatic in nature. Reduction in CFUs may be further impaired in this model owing to the lotion mode of delivery as it is dependent on the diffusion of AVB-Zn into the tissue from a limited surface area. Despite this, treatment with AVB-Zn improved mouse survival relative to the vehicle control and a commercially available bacitracin-Zn ointment. In contrast to the mice in these control groups, no mice in the AVB-Zn treatment group died to overwhelming disease. AVB-Zn then may act to prevent dissemination of bacteria from the infected region that would result in more severe disease. Alternatively, AVB-Zn may alter the virulence profile of MRSA in such a way that reduces disease severity, such as reducing toxin production. Future studies focused on elucidating the mechanism of AVB-Zn will be needed to fully understand its role in preventing severe disease by MRSA. Regardless, AVB-Zn protects against overwhelming disease in a mouse model mimicking a soft tissue infection commonly caused by MRSA.

### Example 3: Discussion

The concept of metallodrugs is ancient and early documentation can be found already in the Ebers papyrus (1500 BCE). As the use of metals and metal salts as treatment for maladies increased over the centuries, it is unsurprising that some efforts were misguided, due to lack of knowledge on mid- and long-term drug toxicities. The use of mercury to treat syphilis, gout, and mental disorders during the 18^{th} and 19^{th} centuries was likely ill advised, and so was the use of arsenic-based potions to treat diabetes and rheumatism. However, metallodrugs are used as antimicrobials, and have certainly become of major importance for the treatment of cancer (e.g. cisplatin, carboplatin). Discovery efforts for antibacterial metallodrugs have focused on copper-compound complexes, as copper by itself is a potent antibacterial. Unfortunately, as copper is redox active, it also causes unspecific toxicity to eukaryotic cells, a problem that has hampered the transition of antibacterial copper-compounds into the clinic. The only non-redox active transition metal, zinc, has not been intensively studied for its potential to be the activating component of metallodrugs. As zinc is not redox active, zinc-antibiotics may have fewer unspecific side effects. This possibility is actually suggested by the common use of the FDA-approved zinc-salt antibiotic bacitracin, or the use of the antifungal zinc-pyrithione for the treatment of dandruff and seborrhoeic dermatitis.

The performed small proof of concept screen (1,600 drugs/compounds) clearly demonstrated that zinc is indeed highly effective in triggering antibacterial compound activities. Consideration of zinc coordination effects expanded the pool of identified hits by >60%. The admittedly serendipitous discovery of zinc-activated avobenzone as a zinc salt with low micromolar activity against various MRSA strains, no preexisting clinical resistance, no resistance generation and demonstration of in vivo activity, in such a small compound library clearly emphasizes the largely unexplored potential of specifically zinc-metallodrugs as antibacterial agents.

Unfortunately, drug screens are usually performed in media, which bear no physiological relevance, and only act as a rich nutritional resource that optimizes bacterial growth, with an exclusive focus on cost-effectiveness. For example, Mueller Hinton broth (MHB) is based on beef extract, casein hydrolysate and starch. These media are not defined, and certainly do not consider physiologically relevant transition metal concentrations. It is established that variation between different media and even between media lots will affect drug discovery. As far as the discovery of zinc-activated metallodrugs is concerned, it has been shown that for example zinc concentrations in just MHB vary between manufacturers and lots (range: 0.2-1.3 μg/mL (3-18 μM)) and these differences have biological and analytical consequences. Differences in zinc concentrations between MHB lots even affect the classification of the antimicrobial susceptibility of metallo-R-lactamase (MBL)-harboring bacteria. In extension, such random and uncontrolled fluctuations in Zn concentrations in undefined media will certainly have effects on drug discovery. As most drug screening conditions that have been or are being used to explore the currently available chemical space did not consider the presence of physiologically relevant concentrations of zinc, these results suggest that re-exploration of available chemical compound libraries for antibacterial zinc-metallodrugs could be a rather promising avenue forward to overcome the current shortage of effective antibiotics.

The antibacterial effect of zinc-bacitracin was described in 1945. More recently, Bohlmann et al. demonstrated that a combination of the ionophore PBT2 and zinc reverses antibiotic resistance, but also can directly kill erythromycin-resistant group A Streptococcus, methicillin-resistant Staphylococcus aureus, and vancomycin-resistant Enterococcus. However, these results were very different from the findings described herein, the antibacterial activity of PTB2 required zinc concentrations between 400-600 μM. This is in stark contrast to the minimal zinc concentrations required for avobenzone activation, which was found to be in the range of 1.25-10 μM, and the observation that higher zinc concentrations would not add additional antibacterial potency to AVB-Zn. Other than PTB2, which is thought to act by interference with zinc homeostasis, the low level requirement for zinc to induce AVB-Zn activity may suggest that Zn here mostly acts to alter the confirmation of avobenzone to allow for better entry into the bacteria, where avobenzone then may begin to form complexes with zinc-binding proteins. Given that 5-10% of the bacterial proteome is thought to require zinc for their activity, this could readily explain the potent effect of AVB-Zn, but also would be consistent with the ability of MRSA to adapt to gradually increasing concentrations of AVB-Zn. Most importantly, if AVB-Zn targets a series of different Zn-dependent proteins it would explain the inability of MRSA to develop resistance by genetic mutations. In a scenario in which intracellular avobenzone were to coordinate with Zn that is associated with different proteins, no mutation to a single gene/protein could rescue the bacteria from AVB-Zn. AVB-Zn could be considered a nutritional immunity mimetic, a concept that describes cellular defense mechanisms which starve bacteria of zinc, an essential micronutrient. The essential question for future studies here would no longer be what the bacterial target of AVB-Zn is, but why AVB-Zn does not affect eukaryotic cells.

Lastly, it needs to be noted that AVB-Zn was specific for MRSA, and had no effect on other tested bacteria such as E. coli or M. tuberculosis. However, as it has become very evident that broad-spectrum antibiotics also have extremely detrimental effects on the human microbiome, a narrow-spectrum antibiotic against MRSA, one of the most dangerous multidrug resistant bacteria, listed as one of the six ESKAPE pathogens, should be a welcomed addition to the disclosed clinical treatment arsenal.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Antimicrobial Resistance C. Global burden of bacterial antimicrobial resistance in 2019: a systematic analysis. Lancet 2022; 399(10325):629-655.
2. Arnaud-Neu F, et al. Synthesis, x-ray crystal structures, and cation-binding properties of alkyl calixaryl esters and ketones, a new family of macrocyclic molecular receptors. Journal of the American Chemical Society 1989; 111(23):8681-91.
3. Bae T et al. Generating a collection of insertion mutations in the Staphylococcus aureus genome using bursa aurealis. Methods Mol Biol 2008; 416:103-116.
4. Bafaro E et al. The emerging role of zinc transporters in cellular homeostasis and cancer. Signal Transduct Target Ther 2017; 2.
5. Bernard R, et al. Resistance to Bacitracin in Bacillus subtilis: Unexpected Requirement of the BceAB ABC Transporter in the Control of Expression of Its Own Structural Genes. Journal of Bacteriology 2007; 189(23):8636-42.
6. Bilinskaya A et al. Variability in Zinc Concentration among Mueller-Hinton Broth Brands: Impact on Antimicrobial Susceptibility Testing of Metallo-beta-Lactamase-Producing Enterobacteriaceae. J Clin Microbiol 2020; 58(12).
7. Bohlmann L et al. Chemical Synergy between Ionophore PBT2 and Zinc Reverses Antibiotic Resistance. mBio 2018; 9(6).
8. Botella H, et al. Mycobacterial P1-Type ATPases Mediate Resistance to Zinc Poisoning in Human Macrophages. Cell Host & Microbe 2011; 10(3):248-59.
9. Brandenburg K S et al. Novel murine model for delayed wound healing using a biological wound dressing with Pseudomonas aeruginosa biofilms. Microb Pathog 2018; 122:30-38.
10. Brazel E B et al. Dysregulation of Streptococcus pneumoniae zinc homeostasis breaks ampicillin resistance in a pneumonia infection model. Cell Rep 2022; 38(2):110202.
11. Brown E D et al. Antibacterial drug discovery in the resistance era. Nature 2016; 529(7586):336-343.
12. Burlov A S, et al. Metal Complexes of Aroyl(acyl)benzoylhydrazones of Aromatic Aldehydes and Ketones: Coordination Modes and Properties. Russian Journal of Coordination Chemistry 2021; 47(7):439-72.
13. Cain B D, et al. Amplification of the bacA gene confers bacitracin resistance to Escherichia coli. Journal of Bacteriology 1993; 175(12):3784-9.
14. Chalker A F, et al. The bacA gene, which determines bacitracin susceptibility in Streptococcus pneumoniae and Staphylococcus aureus, is also required for virulence. Microbiology 2000; 146(7):1547-53.
15. Crawford C L et al. A copper-dependent compound restores ampicillin sensitivity in multidrug-resistant Staphylococcus aureus. Sci Rep 2020; 10(1):8955.
16. Crawford C L et al. Pyrazolopyrimidinones, a novel class of copper-dependent bactericidal antibiotics against multi-drug resistant S. aureus. Metallomics 2019; 11(4):784-798.
17. Cunha T A, et al. Association between zinc and body composition: An integrative review. Journal of Trace Elements in Medicine and Biology 2022; 71:126940.
18. Dalecki A G, et al. Chapter Six—Copper and Antibiotics: Discovery, Modes of Action, and Opportunities for Medicinal Applications. In: Poole RKs (ed). Advances in Microbial Physiology: Academic Press, 2017, 193-260
19. Dalecki A G et al. Combinatorial phenotypic screen uncovers unrecognized family of extended thiourea inhibitors with copper-dependent anti-staphylococcal activity. Metallomics 2016; 8(4):412-421.
20. Dalecki A G, et al. Disulfiram and Copper Ions Kill Mycobacterium tuberculosis in a Synergistic Manner. Antimicrobial Agents and Chemotherapy 2015; 59(8):4835-44.
21. Dalecki A G, et al. High-throughput screening and Bayesian machine learning for copper-dependent inhibitors of Staphylococcus aureus†. Metallomics 2019; 11(3):696-706.
22. Dalecki A G et al. Targeting Biofilm Associated Staphylococcus aureus Using Resazurin Based Drug-susceptibility Assay. J Vis Exp 2016; (111).
23. De Oliveira D M P et al. Rescuing Tetracycline Class Antibiotics for the Treatment of Multidrug-Resistant Acinetobacter baumannii Pulmonary Infection. mBio 2022:e0351721.
24. Eskenazi A et al. Combination of pre-adapted bacteriophage therapy and antibiotics for treatment of fracture-related infection due to pandrug-resistant Klebsiella pneumoniae. Nat Commun 2022; 13(1):302.
25. Festa Richard A, et al. Exploiting Innate Immune Cell Activation of a Copper-Dependent Antimicrobial Agent during Infection. Chemistry & Biology 2014; 21(8):977-87.
26. Fey P D et al. A genetic resource for rapid and comprehensive phenotype screening of nonessential Staphylococcus aureus genes. mBio 2013; 4(1):e00537-00512.
27. Folin M, et al. Zinc content of normal human serum and its correlation with some hematic parameters. Biometals 1994; 7(1):75-9.
28. Gao W et al. Nanomaterials arising amid antibiotic resistance. Nat Rev Microbiol 2021; 19(1):5-6.
29. Ghachi M E, et al. The bacA Gene of Escherichia coli Encodes an Undecaprenyl Pyrophosphate Phosphatase Activity*. Journal of Biological Chemistry 2004; 279(29):30106-13.
30. Giroux E L, et al. A study of zinc distribution in human serum. Bioinorganic Chemistry 1976; 5(3):211-8.
31. Gruet K, et al. Two-Point Cooperative Binding of Ketones by a Metal and by a Neighboring Pendant NH Group. Organometallics 2000; 19(11):2228-32.
32. Hiron A, et al. Bacitracin and nisin resistance in Staphylococcus aureus: a novel pathway involving the BraS/BraR two-component system (SA2417/SA2418) and both the BraD/BraE and VraD/VraE ABC transporters. Molecular Microbiology 2011; 81(3):602-22.
33. Hood M I et al. Nutritional immunity: transition metals at the pathogen-host interface. Nat Rev Microbiol 2012; 10(8):525-537.
34. Huang Y H, et al. Aldehyde and ketone ligands in organometallic complexes and catalysis. Journal of Chemical Education 1988; 65(4):298.
35. Investigators HSGRH. Safety, tolerability, and efficacy of PBT2 in Huntington's disease: a phase 2, randomised, double-blind, placebo-controlled trial. The Lancet Neurology 2015; 14(1):39-47.
36. Johnson B A et al. Bacitracin: A New Antibiotic Produced by a Member of the B. Subtilis Group. Science 1945; 102(2650):376-377.
37. Kehl-Fie T E et al. Nutritional immunity beyond iron: a role for manganese and zinc. Curr Opin Chem Biol 2010; 14(2):218-224.
38. Kennedy A D et al. Epidemic community-associated methicillin-resistant Staphylococcus aureus: recent clonal expansion and diversification. Proc Natl Acad Sci USA 2008; 105(4):1327-1332.
39. Kennedy A D, et al. Complete nucleotide sequence analysis of plasmids in strains of Staphylococcus aureus clone USA300 reveals a high level of identity among isolates with closely related core genome sequences. J Clin Microbiol 2010; 48(12):4504-4511.
40. Kockler J, et al. Chapter Three—Butyl Methoxy Dibenzoylmethane. In: Brittain HGs (ed). Profiles of Drug Substances, Excipients and Related Methodology: Academic Press, 2013, 87-111
41. Lannfelt L, et al. Safety, efficacy, and biomarker findings of PBT2 in targeting Aβ as a modifying therapy for Alzheimer's disease: a phase Ila, double-blind, randomised, placebo-controlled trial. The Lancet Neurology 2008; 7(9):779-86.
42. Lelievre P et al. The Multifaceted Roles of Copper in Cancer: A Trace Metal Element with Dysregulated Metabolism, but Also a Target or a Bullet for Therapy. Cancers (Basel) 2020; 12(12).
43. Lemire J A, et al. Antimicrobial activity of metals: mechanisms, molecular targets and applications. Nature Reviews Microbiology 2013; 11(6):371-84.
44. Lewis K. Antibiotics: Recover the lost art of drug discovery. Nature 2012; 485(7399):439-440.
45. Lewis K. Platforms for antibiotic discovery. Nature Reviews Drug Discovery 2013; 12(5):371-87.
46. Ling L L et al. A new antibiotic kills pathogens without detectable resistance. Nature 2015; 517(7535):455-459.
47. Maret W. The redox biology of redox-inert zinc ions. Free Radical Biology and Medicine 2019; 134:311-26.
48. Ming L J et al. Metal binding and structure-activity relationship of the metalloantibiotic peptide bacitracin. J Inorg Biochem 2002; 91(1):46-58.
49. Murray C J L, et al. Global burden of bacterial antimicrobial resistance in 2019: a systematic analysis. The Lancet 2022; 399(10325):629-55.
50. Ong C-I Y, et al. An Antimicrobial Role for Zinc in Innate Immune Defense Against Group A Streptococcus. The Journal of Infectious Diseases 2014; 209(10):1500-8.
51. Ong C-I Y, et al. Zinc disrupts central carbon metabolism and capsule biosynthesis in Streptococcus pyogenes. Scientific Reports 2015; 5:10799.
52. Ouyang J, et al. The BceABRS four-component system regulates the bacitracin-induced cell envelope stress response in Streptococcus mutans. Antimicrob Agents Chemother 2010; 54(9):3895-906.
53. Radford R J et al. Chelators for investigating zinc metalloneurochemistry. Curr Opin Chem Biol 2013; 17(2):129-136.
54. Ramchandani D et al. Copper depletion modulates mitochondrial oxidative phosphorylation to impair triple negative breast cancer metastasis. Nat Commun 2021; 12(1):7311.
55. Santini C, et al. Advances in Copper Complexes as Anticancer Agents. Chemical Reviews 2014; 114(1):815-62.
56. Shukla R et al. Mode of action of teixobactins in cellular membranes. Nat Commun 2020; 11(1):2848.
57. Silver L L. Challenges of antibacterial discovery. Clin Microbiol Rev 2011; 24(1):71-109.
58. Speer A et al. Copper-boosting compounds: a novel concept for antimycobacterial drug discovery. Antimicrob Agents Chemother 2013; 57(2):1089-1091.
59. Stone K J, et al. Mechanism of Action of Bacitracin: Complexation with Metal Ion and C55-Isoprenyl Pyrophosphate. Proceedings of the National Academy of Sciences 1971; 68(12):3223-7.
60. Totten A H, et al. Differential Susceptibility of Mycoplasma and Ureaplasma Species to Compound-Enhanced Copper Toxicity. Frontiers in Microbiology 2019; 10 71
61. Wagner D, et al. Elemental Analysis of Mycobacterium avium, Mycobacterium tuberculosis, and Mycobacterium smegmatis-Containing Phagosomes Indicates Pathogen-Induced Microenvironments within the Host Cell's Endosomal System. The Journal of Immunology 2005; 174(3):1491 72
62. Wang F et al. Turning tumor-promoting copper into an anti-cancer weapon via high-throughput chemistry. Curr Med Chem 2010; 17(25):2685-2698.
63. Yoshida Y, et al. Bacitracin sensing and resistance in Staphylococcus aureus. FEMS Microbiology Letters 2011; 320(1):33-9.

1. A method for treating or preventing a bacterial infection in a subject, the method comprising administering a composition comprising a transition metal and avobenzone to the subject.

2. The method of claim 1, wherein the transition metal comprises copper, zinc, manganese, nickel, cobalt, silver, iron, another transition metal, or any combination thereof.

3. The method of claim 1, wherein the transition metal is in a +1 or +2 oxidation state.

4.-8. (canceled)

9. The method of claim 1, wherein the composition is administered topically and is formulated as a cream, an ointment, a paste, a gel, a lotion, a milk, a suspension, an aerosol, a non-aerosol spray, a foam, a dusting powder, a pad, a patch, or any combination thereof.

10. The method of claim 1, wherein the composition is administered to a site comprising the skin of a subject, a wound, a burn, a surgical incision, or any combination thereof.

11. The method of claim 1, wherein the composition is administered orally and is formulated as tablets, capsules, pills, powder, granules, a suspension, a syrup, an emulsion, or any combination thereof.

12. The method of claim 1, wherein the bacterial infection is caused by a Gram-positive bacterium.

13. The method of claim 12, wherein the Gram-positive bacterium is methicillin-resistant Staphylococcus aureus (MRSA).

14. The method of claim 12, wherein the Gram-positive bacterium is resistant to bacitracin.

15. The method of claim 1, further comprising administering an antibiotic to the subject.

16. The method of claim 15, wherein the antibiotic comprises amoxicillin, clavulanic acid, ampicillin, sulfobactam, penicillin, vancomycin, ceftriaxone, daptomycin, ciprofloxacin, levofloxacin, ofloxacin, clindamycin, erythromycin, gentamicin, tetracycline, sulfamethoxazole, trimethoprim, another antibiotic, or any combination thereof.

17.-19. (canceled)

20. The method of claim 1, wherein the composition comprises from about 0.5 μM to about 100 μM Zn(II).

21. The method of claim 1, wherein the composition comprises from about 1 μM to about 25 mM avobenzone.

22. The method of claim 20, wherein the composition comprises about 10 μM Zn(II) and about 25 mM avobenzone.

23.-24. (canceled)

25. A composition comprising avobenzone, a transition metal salt, and at least one carrier or excipient, wherein the at least one carrier or excipient comprises polyethylene glycol (PEG), water, an emulsifier, nanoparticles, a caged-delivery system, or any combination thereof.

26. The composition of claim 25, wherein the transition metal salt comprises CuSO₄, ZnSO₄, ZnCl₂, zinc lactate, or a combination thereof.

27. The composition of claim 26, wherein the composition comprises from about 0.5 μM to about 100 μM ZnSO₄.

28. The composition of claim 26, wherein the composition comprises from about 1 μM to about 25 mM avobenzone.

29. The composition of claim 26, wherein the composition comprises about 10 μM ZnSO₄ and about 25 mM avobenzone.

30.-31. (canceled)

32. The composition of claim 25, wherein the composition is formulated as a cream, an ointment, a paste, a gel, a lotion, a milk, a suspension, an aerosol, a non-aerosol spray, a foam, a dusting powder, a pad, a patch, tablets, capsules, pills, powder, granules, a suspension, a syrup, an emulsion, or any combination thereof.
